(19) United States
(12) Patent Application Publication
(10) Pub. No.: US 2021/0008029 A1
(43) Pub. Date: Jan. 14, 2021

(54) SEMISOLID DOSAGE FORMS FOR OPHTHALMIC DELIVERY OF ANTIOXIDANTS

(71) Applicant: Tathagata DUTTA, BERHAMPORE (IN)

(72) Inventor: Tathagata DUTTA, BERHAMPORE (IN)

(21) Appl. No.: 16/982,770

(22) PCT Filed: Nov. 21, 2018
(86) PCT No.: PCT/IN2018/050769

§ 371 Date: Sep. 21, 2020

(30) Foreign Application Priority Data
Oct. 3, 2018 (IN) 201811037373

(51) Int. Cl. A61K 31/355; A61K 9/06; A61K 9/00; A61K 33/04; A61K 31/047; A61K 47/44; A61K 47/34; A61K 47/02

(52) U.S. Cl. CPC A61K 31/355; A61K 9/06; A61K 9/0048; A61K 47/02; A61K 31/047; A61K 47/44; A61K 47/34; A61K 33/04

(57) ABSTRACT

A stable semisolid dosage form (Ointment, Cream or Gel) for ocular delivery of antioxidants, comprising one or more antioxidants as active pharmaceutical ingredient, at least one semisolid base (either Ointment, Gel or a Cream base), and at least one pharmaceutically acceptable excipient, wherein the pH is in the range of about 4 to 8. The present invention provides an emulsion composition that provides release of the active agent in sustained manner. The present invention provides a steady concentration of antioxidants in the aqueous & vitreous humor. The present invention increases the macular pigment optical density (MPOD), for better management of Age Related Macular Degeneration. The present invention provides a semi solid dosage in three different from ointment, cream and gel to treat medical conditions of the eye.

### FIELD OF THE INVENTION

The present invention relates to a method to treat medical conditions of the eye by drug delivery dosage forms. More specifically the present invention relates to semisolid drug delivery dosage forms of antioxidants for drug delivery within the eye.

### BACKGROUND

Age-related macular degeneration (AMD) is the most prevalent cause of irreversible blindness in developed countries. High-dose supplementation with beta carotene, vitamins C and E, and zinc shown to slow the progression of AMD. Age related macular degeneration (ARMD) is a severe problem of the eye which accounts for the loss of vision of huge number of elderly population across the globe. The disease which starts as a mild innocent problem of the eye, including occasional floaters and black dots in front of the eye gradually progresses to the loss of peripheral vision to complete loss of vision. Age-related macular degeneration (AMD) is leading cause of blindness worldwide, is a bilateral ocular condition that affects the central area of retina known as the macula. Antioxidants are well known to slow down the progression of the ARMD disease. Antioxidant vitamins help to retard some of the destructive processes in the retina and the retinal pigment epithelium that lead to age-related degeneration of the macula.

U.S. Pat. No. 5,496,811A discloses an ocular drug delivery vehicle of an oil-in-water submicron emulsion comprising about 0.5 to 50% of a first component of an oil, about 0.1 to 10% of a second component of an emulsifier, about 0.05 to 5% of a non-ionic surfactant and an aqueous component, with the mean droplet size being in the submicron range, i.e., below about 0.5 pm and preferably between about 0.1 and 0.3 pm. Also, topical pharmaceutical compositions containing a drug such as an anti-glaucoma drug, beta adrenergic blocker or other autonomic system drug, a local anesthetic, a steroid, a non-steroidal anti-inflammatory drug, an antibiotic drug, an antifungal drug, an antiviral drug or combinations thereof and the vehicle described above. Methods of administering such vehicles or compositions to the eye of a patient while reducing irritation thereof and providing increased bioavailability of the drug.

US20060182783A1 discloses a biocompatible intraocular drug delivery systems include an anti-angiogenic macromolecular therapeutic agent and a polymeric component in the form of an implant, a micro particle, a plurality of implants or micro particles, and combinations thereof. The therapeutic agent is released in a biologically active form, for example, the therapeutic agent may retain its three dimensional structure when released into an eye of a patient, or the therapeutic agent may have an altered three dimensional structure but retain its therapeutic activity. The therapeutic agent contains a component selected from the group consisting of anti-angiogenesis peptides and nucleic acid agents. The implants may be placed in an eye to treat or reduce the occurrence of one or more ocular conditions, such as retinal damage, including glaucoma and proliferative vitreoretinopathy among others.

US20050271705A1 discloses a biocompatible intraocular implant include a retinoid component and a biodegradable polymer that is effective to facilitate release of the retinoid component into an eye for an extended period of time. The therapeutic agents of the implants may be associated with a biodegradable polymer matrix, such as a matrix that is substantially free of a polyvinyl alcohol. The implants may be placed in an eye to treat or reduce the occurrence of one or more ocular conditions, such as retinal damage, including glaucoma and proliferative vitreoretinopathy.

In the prior art the existing invention several formulations available in the market containing single or combination of antioxidants like Alpha tocopherol, lutein, zeaxanthin, Beta Carotene, Selenium, etc. for oral administration for slowing down the progression of the disease. There are several drawbacks with orally administered drugs or bioactive as the drug does not reach the eye at appropriate concentrations and has either none or very poor pharmacological action in the eye when administered through oral route. The need therefore has arisen to develop a topical formulation of antioxidants for ocular administration that will release the drug in a sustained manner in the eye that on repeated administration will lead to a steady concentration of antioxidants in the aqueous and vitreous humor thereby increasing the macular pigment optical density (MPOD), which will be helpful for better management of Age Related Macular Degeneration.

### Objective of the Invention

The main objective of the present invention is to provide a method to treat medical conditions of the eye by drug delivery dosage forms.

Another objective of the present invention is to provide a semisolid drug delivery dosage of antioxidants for drug delivery within the eye.

Yet another objective of the present invention is to provide an emulsion composition that provides release of the active agent in sustained manner

Yet another objective of the present invention is to provide a steady concentration of antioxidants in the aqueous & vitreous humor.

Yet another objective of the present invention is to increase the macular pigment optical density (MPOD), for better management of Age Related Macular Degeneration.

Yet another objective of the present invention is to provide a semi solid dosage in three different from ointment, cream and gel to treat medical conditions of the eye.

Further objectives and features of the present invention will become apparent from the detailed description provided herein below, in which various embodiments of the disclosed present invention are illustrated by way of example and appropriate reference.

### SUMMARY

Accordingly, the invention provides new topical drug delivery system that releases the active agent in a sustained manner to provide the desired therapeutic effects, and methods of making such systems. The topical drug delivery system of the invention as disclosed herein is semisolid dosage form of either one or combination of antioxidants. The emulsion composition as disclosed herein provides release of the active agent in sustained manner and repeated administration of the formulation several times a day will lead to a steady concentration of antioxidants in the aqueous &vitreous humor increasing the macular pigment optical density (MPOD), which will be helpful for better management of Age Related Macular Degeneration.

The semisolid dosage composition as disclosed herein is a stable semisolid dosage form formulation of one or more antioxidants, at least one oily base from vegetable, mineral, polymeric andnimal origin, wherein the pH range is from about 4 to 8. The concentration of the antioxidant in the emulsion composition as disclosed herein is in the range of about 0.0003%-0.3% w/v and the antioxidant is selected from the group comprising of lutein, zeaxanthin, tocopherol, beta carotene, selenium or a combination thereof.

The semisolid base is either an ointment base like white soft paraffin, petrolatum, lanoline, bees wax, carnauba wax, etc. or a combination thereof, or an emulsion base comprising of an oil, where the oil is a vegetable oil or mineral oil or animal oil or a combination thereof or a gel forming polymer like hydroxypropyl methyl cellulose (HPMC), carboxy methyl cellulose (CMC), povidone, cross povidone, carbopol, polyethylene glycol, polypropylene glycol, etc. The Formulation may also contain an antimicrobial preservative like methyl paraben, propyl paraben, benzalkonium chloride, phenylmercuric nitrate, etc., and a tonicity adjusting agent.

The vegetable oil is selected from the group comprising of Castor Oil, Cotton seed Oil, Peanut Oil, Coconut oil, Rice bran oil, Sunflower oil, Sesame oil, soya bean oil, Flax oil, Canola oil, Olive oil, Mustard Oil, Jojoba oil or a combination thereof. The animal oil is selected from the group comprising of fish oil, shark liver oil, cod liver oil or a combination thereof. The mineral oil is liquid paraffin.

The surfactant is selected from the group comprising of Polyoxyethylated nonionic surfactants like polysorbate80, polysorbate 60, polysorbate, 40, polysorbate 20, cremophors, tyloxapols, poloxamers, benzalkonium chloride, benzethonium chloride, cetyl alcohol, carbomer, cholesterol, cocamidopropyl betaine, glyceryl monostearate, lanolin alcohols, lauralkonium chlorides, n lauroylsarcosine, nonoxynol 9, octoxynol 40, polyoxyl castor oil, polyoxyl 40 hydrgenated castor oil, polyoxyl 40 stearate, sorbitanmonolaureate, sorbitan monooleate, sorbitanmonopalmitate, polyoxyethylene (2) steryl ether, polyoxyethylene (2) cetyl ether, polyoxyethylene (2) oleyl ether, polyeoxyethyllene (2) nonylphenylether, polyoxyethylene (2) isooctylphenyl ether, polyoxyletheylene (4) lauryl ether, polyoxylethylene (5) isooctylphenylether or a combination thereof.

The semisolid dosage form composition as described herein further comprises at least one pH adjusting agent, at least one buffering agent, at least one osmolarity control agent and at least one antimicrobial preservative.

The pH adjusting agent selected from the group comprising of Hydrochloric Acid, Sodium Hydroxide, Sulphuric Acid, Sodium Sulphate, Acetic Acid, Sodium Citrate, Ammonium Hydroxide, Citric Acid, Diethanolamine, Nitric Acid, Phosphoric Acid or a combination thereof.

The buffering agent selected from the group comprising of Acetic Acid, Boric Acid, Citric Acid, Phosphoric Acid, Potassium Acetate, Potassium Phosphate, Potassium Sulphate, Potassium Sorbate, Sodium Acetate, Sodium borate, Sodium Carbonate, Sodium Citrate, Sodium Phosphate, Sorbic Acid, Tromethamine or a combination thereof.

The osmolarity control agent selected from the group comprising of Sodium Chloride, Sodium Sulphate, Sodium Nitrate, Sorbitol, Mannitol, Calcium Chloride, Glycerin, Magnesium Chloride, PEG 300, PEG 400, Potassium Chloride, Propylene Glycol or a combination thereof.

The antimicrobial preservative selected from the group comprising of Quaternary ammonium compounds selected from Benzalkonium Chloride, Benzethonium chloride, Benzododecinium bromide or Polyquatermium-1; or Acid/Base compounds selected from Boric acid, sodium acetate or sodium borate; or Alcohols selected from chlorobutanol or Phenylethyl alcohol; or Organic Mercuric compounds selected from Phenyl mercuric acetate, Phenyl mercuric nitrate or Thimerosal; or Parabens selected from methyl paraben or Propyl Paraben; or Oxidizing agent sodium chlorite; or Metal salt Zinc Chloride or a combination thereof.

The main advantage of the present invention is that present invention provides a method to treat medical conditions of the eye by drug delivery dosage forms.

Another advantage of the present invention is that the present invention provides a semisolid drug delivery dosage of antioxidants for drug delivery within the eye.

Yet another advantage of the present invention is that the present invention provides an emulsion composition that provides release of the active agent in sustained manner

Yet another advantage of the present invention is the present invention provides a steady concentration of antioxidants in the aqueous & vitreous humor.

Yet another advantage of the present invention is that the present invention increases the macular pigment optical density (MPOD), for better management of Age Related Macular Degeneration.

Yet another advantage of the present invention is that the present invention provides a semi solid dosage in three different from ointment, cream and gel to treat medical conditions of the eye.

Further advantage and features of the present invention will become apparent from the detailed description provided herein below, in which various embodiments of the disclosed present invention are illustrated by way of example and appropriate reference.

### DETAILED DESCRIPTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### Definition

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended. The term “comprising” is used interchangeably used by the terms “having” or “containing”.

Reference throughout this document to “one embodiment”, “certain embodiments”, “an embodiment”, “another embodiment”, and “yet another embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics are combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, the term “one or more” generally refers to, but not limited to, singular as well as plural form of the term.

The drawings featured in the figures are for the purpose of illustrating certain convenient embodiments of the present invention, and are not to be considered as limitation there to. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

The present invention includes a stable semisolid dosage form for ophthalmic delivery of antioxidants. The semisolid dosage includes an antioxidant as an active pharmaceutical ingredient, and a pharmaceutically acceptable excipient. Herein pH is in the range of about 4 to 8. As used herein, the term “semisolid dosage” refers to but not limited to, semisolid dosage pharmaceutical system comprise a body product that applied to skin membrane tends to alleviate a pathological condition against harmful environment. As used herein, the term “antioxidant” refers to but not limited to, the substances that can prevent damage to cells caused by free radicals, unstable molecules that the body produces as a reaction to environmental and other pressures. As used herein, the term “pH” refers to but not limited to, the measure of acidity and alkalinity of an aqueous solution. In an embodiment, the antioxidant is selected as an active pharmaceutical ingredient and the antioxidant is of different groups including, but not limited to, group of lutein, zeaxanthin, tocopherol, beta carotene, selenium and combination thereof. In an embodiment, the concentration of the antioxidant is in the range of 0.0003%-0.3% w/v. In an embodiment, the stable semisolid dosage form includes a surfactant, but not limited to, a group of polyoxyethylated nonionic surfactants like polysorbate80, polysorbate 60, polysorbate, 40, polysorbate 20, cremophors, tyloxapols, poloxamers, benzalkonium chloride, benzethonium chloride, cetyl alcohol, carbomer, cholesterol, cocamidopropyl betaine, glyceryl monostearate, lanolin alcohols, lauralkonium chlorides, n lauroylsarcosine, nonoxynol 9, octoxynol 40, polyoxyl castor oil, polyoxyl 40 hydrgenated castor oil. polyoxyl 40 stearate, sorbitanmonolaureate, sorbitan monooleate, sorbitanmonopalmitate, polyoxyethylene (2) steryl ether, polyoxyethylene (2) cetyl ether, polyoxyethylene (2) oleyl ether, polyeoxyethyllene (2) nonylphenylether, polyoxyethylene (2) isooctylphenyl ether, polyoxyletheylene (4) lauryl ether, polyoxylethylene (5) isooctylphenylether and combination thereof. In an embodiment, the stable semisolid dosage form includes a pH adjusting agent, a buffering agent, an osmolarity control agent and an antimicrobial preservative. As used herein, the term “pH adjusting agent” refers to but not limited to, an agent that adjust the pH of the solution in acidic and basic medium. As used herein, the term “buffering agent” refers to but not limited to, a weak acid and base used to maintain the acidity (pH) of a solution in acid and base. The buffering agent prevents rapid change in pH when acids and bases are added to the solution. The buffering agents have soluble than others. As used herein, the term “osmolarity agent” refers to but not limited to, an agents used in the regulation of encapsulated cell behavior to provide a safer and more predictable delivery of therapeutics. As used herein, the term “antimicrobial agent” refers to but not limited to, an agent that kills microorganisms and stops their growth. In an embodiment, the pH adjusting agent is of different groups including, but not limited to, a group of hydrochloric acid, sodium hydroxide, sulphuric acid, sodium sulphate, acetic acid, sodium citrate, ammonium hydroxide, citric acid, diethanolamine, nitric acid, phosphoric acid and combination thereof. In an embodiment, the buffering agent is of different groups including, but not limited to, a group of acetic acid, boric acid, citric acid, phosphoric acid, potassium acetate, potassium phosphate, potassium sulphate, potassium sorbate, sodium acetate, sodium borate, sodium carbonate, sodium citrate, sodium phosphate, sorbic acid, tromethamine and combination thereof. In an embodiment, the osmolarity control agent is of different groups including, but not limited to, a group of sodium chloride, sodium sulphate, sodium nitrate, sorbitol, mannitol, calcium chloride, glycerin, magnesium chloride, PEG 300, PEG 400, potassium chloride, propylene glycol or a combination thereof. In an embodiment, the antimicrobial preservative is of different groups including, but not limited to, a group of quaternary ammonium compounds selected from benzalkonium chloride, benzethonium chloride, benzododecinium bromide, Polyquatermium-1 acid/base compounds selected from boric acid, sodium acetate or sodium borate/alcohols selected from chlorobutanol, phenylethyl alcohol, and organic mercuric compounds selected from phenyl mercuric acetate, phenyl mercuric nitrate or thimerosal; or parabens selected from methyl paraben, propyl, paraben oxidizing agent sodium chlorite; and metal salt zinc chloride and combination thereof. In an embodiment, the stable semisolid dosage includes an ophthalmic ointments, an ophthalmic gel and an ophthalmic creams. As used herein, the term “ophthalmic” refers to but not limited to, relating to the eye and its diseases. In an embodiment, the ophthalmic ointment includes different types of ointment base including, but not limited to, a group of white soft paraffin, petrolatum, lanoline, bees wax, carnauba wax and combination thereof. In an embodiment, the ophthalmic gel includes different types of gel forming polymer including, but not limited to, a group of hydroxypropyl methyl cellulose (HPMC), carboxy methyl cellulose (CMC), povidone, cross povidone, carbopol, polyethylene glycol, polypropylene glycol and combination thereof. In an embodiment, the ophthalmic cream (oil-in-water ophthalmic emulsion) includes different types of oil including, but not limited to, a vegetable oil, a mineral oil and an animal oil. In an embodiment, the vegetable oil includes different types of oil including, but not limited to, a group of castor oil, cotton seed oil, peanut oil, coconut oil, rice bran oil, sunflower oil, sesame oil, soya bean oil, flax oil, canola oil, olive oil, mustard oil, jojoba oil and combination thereof. In an embodiment, the animal oil includes different types of oil including, but not limited to, a group of fish oil, shark liver oil, cod liver oil and combination thereof. In an embodiment, the mineral oil includes different types of oil including, but not limited to, a group of liquid paraffin and white mineral oil and combination thereof.

### Example: 1

### Lutein+Zeaxanthin Ophthalmic Emulsion

| S. No | Ingredient | mg/mL | |
|---|---|---|---|
| 1 | Lutein | 3 | mg |
| 2 | Zeaxanthin | 3 | mg |
| 3 | White Paraffin wax | 39 | mg |
| 6 | Benzalkonium Chloride | 1 | mg |

| | | |
|---|---|---|
| 9 | Hydrochloric acid/sodium hydroxide | q.s |

In the preferred embodiment, the present invention includes process of the ophthalmic ointment that includes, white paraffin wax was heated to about 70° C. and to it was added lutein & zeaxanthin and stirred to completely dissolve the same and to it was added benzalkonium chloride. Sterile purified water was heated to about 70° C., and to it polysorbate 80, Sodium citrate, Benzalkonium Chloride and HPMC was added and mixed to form the aqueous phase. The ointment was finely divided in a high shear mechanicalhomogenizer sterilized by filtration through 0.22 μm membrane filter to form the sterile ophthalmic ointment of antioxidants, which is cooled to solidify, filled into containers aseptically and sealed. In another embodiment, the ophthalmic semisolid dosage form is an emulsion (Ophthalmic Cream) includes the antioxidants lutein, zeaxanthin, alpha tocopherol, and selenium and pharmaceutical excipients.

### Example: 2

### Lutein, Zeaxanthin, Alpha Tocopherol & Selenium Ophthalmic Cream (Oil in Water Emulsion Base)

| S. No | Ingredient | mg/mL | |
|---|---|---|---|
| 1 | Lutein | 3 | mg |
| 2 | Zeaxanthin | 6 | mg |
| 3 | Alpha Tocopherol | 0.5 | mg |
| 4 | Selenium | 0.01 | mg |
| 5 | Peanut Oil | 81 | mg |
| 6 | Poloxamer 188 | 56 | mg |
| 7 | Polyethylene Glycol (PEG) 400 | 20 | mg |
| 8 | Sodium chloride | 9 | mg |
| 9 | Phenyl mercuric nitrate | 2 | mg |

| | | |
|---|---|---|
| 10 | Water for Injection | q.s |
| 11 | Sodium Hydroxide/Hydrochloric Acid | q.s |

In the preferred embodiment the present invention includes, process of Ophthalmic Cream, that includes, Peanut oil was heated to about 70° C. and to it was added Lutein, Zeaxanthin and alpha tocopherol and stirred to completely dissolve the same in the oil phase. Sterile purified water was heated to about 70° C. ,and to it Selenium, Poloxamer 188, Polyethylene Glycol (PEG) 400, Sodium Chloride, and Phenyl mercuric nitrate was added and mixed to form the aqueous phase. The oil phase was added while stiffing the aqueous phase with a high shear mixer to form the emulsion. This crude emulsion was finely divided in a high pressure homogenizer to form a stable cream of antioxidants and the volume was made up with sufficient quantity of water for injection and the pH was adjusted using either sodium hydroxide/hydrochloric acid and sterilized by filtration through 0.22 μm membrane filter to form the sterile ophthalmic emulsion of antioxidants, which is filled into containers aseptically and sealed.

In another embodiment of the invention, the ophthalmic gel comprises lutein, alpha tocopherol and pharmaceutical excipients.

### Example: 3

### Lutein+Alpha Tocopherol Ophthalmic Emulsion

| S. No | Ingredient | mg/mL | |
|---|---|---|---|
| 1 | Lutein | 2 | mg |
| 2 | Alpha Tocopherol | 0.3 | mg |
| 3 | Hydroxy Propyl Methyl Cellulose (HPMC) | 60 | mg |
| 6 | Sodium Citrate | 0.2 | mg |
| 7 | Citric Acid | 0.6 | mg |
| 8 | Benzalkonium Chloride | 1 | mg |

| | | |
|---|---|---|
| 9 | Hydrochloric Acid/Sodium Hydroxide | q.s |
| 10 | Water for Injection | q.s |

In the preferred embodiment the present invention includes, process of Ophthalmic gel, that includes, Water for Injection was heated to about 70° C. and to it was added HPMC, sodium citrate, citric acid, benzalkonium chloride and stirred to completely dissolve. to this was added lutein and alpha tocopherol along with high shear homigebization. The volume was made up with sufficient quantity of water for injection and the pH was adjusted using either sodium hydroxide/hydrochloric acid under homogenization and sterilized by filtration through 0.22 μm membrane filter. The mixture was cooled gradually till the semisolid ophthalmic gel of antioxidants is formed which is filled into containers aseptically and sealed.

Further objectives, advantages and features of the present invention will become apparent from the detailed description provided herein below, in which various embodiments of the disclosed present invention are illustrated by way of example and appropriate reference to accompanying drawings. Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

1. A stable semisolid dosage form for ophthalmic delivery of antioxidants, the semisolid dosage comprising:
an at least one antioxidant as an active pharmaceutical ingredient; and
an at least one pharmaceutically acceptable excipient;
wherein pH is in the range of about 4 to 8.

2. As claimed in claim 1, wherein the stable semisolid dosage form comprising a surfactant that is selected from a group of polyoxyethylated nonionic surfactants like polysorbate 80, polysorbate 60, polysorbate, 40, polysorbate 20, cremophors, tyloxapols, poloxamers, benzalkonium chloride, benzethonium chloride, cetyl alcohol, carbomer, cholesterol, cocamidopropyl betaine, glyceryl monostearate, lanolin alcohols, lauralkonium chlorides, n lauroylsarcosine, nonoxynol 9, octoxynol 40, polyoxyl 35 castor oil, polyoxyl 40 hydrgenated castor oil. polyoxyl 40 stearate, sorbitanmonolaureate, sorbitan monooleate, sorbitanmonopalmitate, polyoxyethylene (2) steryl ether, polyoxyethylene (2) cetyl ether, polyoxyethylene (2) oleyl ether, polyeoxyethyllene (2) nonylphenylether, polyoxyethylene (2) isooctylphenyl ether, polyoxyletheylene (4) lauryl ether, polyoxylethylene (5) isooctylphenylether and combination thereof.

3. As claimed in claim 1, wherein the stable semisolid dosage form comprising an pH adjusting agent selected from a group of hydrochloric acid, sodium hydroxide, sulphuric acid, sodium sulphate, acetic acid, sodium citrate, ammonium hydroxide, citric acid, diethanol amine, nitric acid, phosphoric acid and combination thereof.

4. As claimed in claim 1, wherein the stable semisolid dosage form comprising an buffering agent selected from a group of acetic acid, boric acid, citric acid, phosphoric acid, potassium acetate, potassium phosphate, potassium sulphate, potassium sorbate, sodium acetate, sodium borate, sodium carbonate, sodium citrate, sodium phosphate, sorbic acid, tromethamine and combination thereof.

5. As claimed in claim 1, the stable semisolid dosage form comprising an osmolarity control agent selected from a group of sodium chloride, sodium sulphate, sodium nitrate, sorbitol, mannitol, calcium chloride, glycerin, magnesium chloride, peg 300, peg 400, potassium chloride, propylene glycol or a combination thereof.

6. As claimed in claim 1, the stable semisolid dosage form comprising at least one antimicrobial preservative selected from a group of quaternary ammonium compounds selected from benzalkonium chloride, benzethonium chloride, benzododecinium bromide, polyquatermium-1 acid/base compounds selected from boric acid, sodium acetate or sodium borate/alcohols selected from chlorobutanol, phenylethyl alcohol, and organic mercuric compounds selected from phenyl mercuric acetate, phenyl mercuric nitrate or thimerosal; or parabens selected from methyl paraben, propyl, paraben oxidizing agent sodium chlorite; and metal salt zinc chloride and combination thereof.

7. As claimed in claim 1, wherein the at least one antioxidant is selected from the group of lutein, zeaxanthin, tocopherol, beta carotene, selenium and combination thereof.

8. As claimed in claim 1, wherein the concentration of the at least one antioxidant is in the range of 0.0003%-0.3% w/v.

9. As claimed in claim 1, wherein the stable semisolid dosage form is selected from an ophthalmic ointments, an ophthalmic gel and an ophthalmic creams.

10. As claimed in claim 9, wherein the ophthalmic ointment comprising ointment base is selected from a group of white soft paraffin, petrolatum, lanoline, bees wax, carnauba wax and combination thereof.

11. As claimed in claim 9, wherein the ophthalmic gel comprising gel forming polymer is selected from a group of hydroxypropyl methyl cellulose (HPMC), carboxy methyl cellulose (CMC), povidone, cross povidone, carbopol, polyethylene glycol, polypropylene glycol and combination thereof.

12. As claimed in claim 9, wherein the ophthalmic cream (oil-in-water ophthalmic emulsion) is selected from a vegetable oil, a mineral oil and an animal oil.

13. As claimed in claim 12, wherein the vegetable oil is selected from the group of castor oil, cotton seed oil, peanut oil, coconut oil, rice bran oil, sunflower oil, sesame oil, soya bean oil, flax oil, canola oil, olive oil, mustard oil, jojoba oil and combination thereof.

14. As claimed in claim 12, wherein the animal oil is selected from the group of fish oil, shark liver oil, cod liver oil and combination thereof.

15. As claimed in claim 12, wherein the mineral oil is selected from the group of liquid paraffin and white mineral oil and combination thereof.
